(19) United States
(12) Patent Application Publication
(10) Pub. No.: US 2024/0091009 A1
(43) Pub. Date: Mar. 21, 2024

(54) ANNULOPLASTY APPARATUS, PROCEDURAL APPARATUS AND ANNULOPLASTY SYSTEM

(71) Applicant: SILARA MEDTECH INC., Santa Rosa, CA (US)

(72) Inventors: Trevor M. GREENAN, Santa Rosa, CA (US); Travis ROWE, Santa Rosa, CA (US)

(21) Appl. No.: 18/255,067

(22) PCT Filed: Nov. 29, 2021
(86) PCT No.: PCT/US2021/060961

§ 371 Date: May 30, 2023

Related U.S. Application Data
(60) Provisional application No. 63/119,551, filed on Nov. 30, 2020.

(51) Int. Cl. A61F 2/24

(52) U.S. Cl. CPC A61F 2/2445; A61F 2/2466; A61F 2210/0014; A61F 2220/0016

(57) ABSTRACT

The present disclosure relates to an annuloplasty apparatus configured to be implanted into the body of a subject, the annuloplasty apparatus comprising: a contractile bridging element; a bar member connected to the contractible bridging element, the bar member being provided with a rotatable connection mechanism; and a tissue anchor configured to secure the bar member to the annulus tissue by the rotatable connection mechanism, and comprising: a head portion; and a helical tissue coupling element, the proximal end of which is fixed to the head portion, wherein the helical tissue coupling element is configured to be driven into the annulus tissue by rotation, wherein the rotatable connection mechanism is at least partially configured to be rotatable relative to the bar member, thereby allowing the helical tissue coupling element to rotate further relative to the bar member when the proximal end of the helical tissue coupling element is in contact with the rotatable connection mechanism. In this way, the gap between the bar member and the annulus

### CROSS-REFERENCE TO RELATED APPLICATIONS

This application claims priority to U.S. Provisional Application No. 63/119,551, filed on Nov. 30, 2020, which is herein incorporated by reference in its entirety.

### TECHNICAL FIELD

The present disclosure relates to the field of annuloplasty, and in particular, to an annuloplasty apparatus, a procedural apparatus for performing an annuloplasty, and an annuloplasty system comprising the annuloplasty apparatus and the procedural apparatus.

### BACKGROUND

There are currently many devices and methods for treating mitral regurgitation. These devices and methods primarily include replacement or repair of the mitral valve. Replacement of the mitral valve is usually performed through transapical or transseptal procedures. There are usually four types of mitral valve repair: valve leaflet clip; direct annuloplasty; indirect annuloplasty; and chordae tendineae repair. Both direct and indirect annuloplasty involve reshaping the mitral annulus and/or left ventricle of a subject so that the anterior and posterior leaflets are properly closed to prevent regurgitation by eliminating mitral insufficiency. For some annuloplasty applications, a shaping ring is implanted in the vicinity of the mitral annulus, the purpose of which is to reduce the circumference of the mitral annulus so that the anterior and posterior leaflets are brought closer to prevent regurgitation. The tricuspid valve can also be replaced or repaired by similar devices and methods.

In some techniques for repairing the mitral valve, it is necessary to fix the implant in the human body with an anchor. In the prior art, in order to deliver the anchor to a target location accurately, it is necessary to sleeve the anchor over a guide, and an anchor deployment tool delivers the anchor to a desired location in the body and anchors the implant under the guidance of the guide. The anchor deployment tool needs to reliably couple, guide, and release the anchoring element in the proper procedure time.

In fixing the implant, the anchoring element may be coupled to the implant in a number of ways. One way is to provide a crossbar at the anchoring location of the implant, and a helical tissue coupling element of the anchoring element may be advanced around the crossbar into the tissue until a proximal end of the helical tissue coupling element is in contact with the crossbar. In some cases, after the anchoring is completed, it cannot ensure that the implant is in tight contact with the tissue.

### SUMMARY

An object of the present disclosure is to provide an annuloplasty apparatus capable of alleviating or eliminating the above-mentioned technical drawbacks.

Another object of the present disclosure is to provide a procedural apparatus for facilitating implantation of a tissue anchor into annulus tissue.

According to a first aspect of the present disclosure, there is provided an annuloplasty apparatus configured to be implanted into the body of a subject, the annuloplasty apparatus comprising: a contractile bridging element; a bar member connected to the contractible bridging element, the bar member being provided with a rotatable connection mechanism; and a tissue anchor configured to secure the bar member to the annulus tissue by the rotatable connection mechanism, and comprising: a head portion; and a helical tissue coupling element, the proximal end of which is fixed to the head portion, wherein the helical tissue coupling element is configured to be driven into the annulus tissue by rotation, wherein the rotatable connection mechanism is at least partially configured to be rotatable relative to the bar member, thereby allowing the helical tissue coupling element to rotate further relative to the bar member when the proximal end of the helical tissue coupling element is in contact with the rotatable connection mechanism.

According to a second aspect of the present disclosure, there is provided a procedural apparatus for performing an annuloplasty, the procedural apparatus comprising: a tissue anchor having a longitudinal center axis and being configured to define a passage extending through the tissue anchor along the longitudinal central axis, wherein the tissue anchor includes a helical tissue coupling element having a proximal end and a distal end, the helical tissue anchoring element defining a portion of the passage of the tissue anchoring element; an anchor deployment tool which includes: a catheter having a catheter distal end; and a rotation driving body having a proximal end, a distal end, and a longitudinal through hole extending from the proximal end to the distal end thereof, wherein the proximal end of the rotation driving body is connected to the catheter distal end; and an elongate guide configured to be able to extend through the catheter and the longitudinal through hole of the rotation driving body, wherein the rotation driving body is configured to guide the tissue anchor by extending within the passage of the helical tissue coupling element during anchoring the tissue anchor to annulus tissue by rotating the tissue anchor.

According to a third aspect of the present disclosure, there is provided an annuloplasty system, comprising: an annuloplasty apparatus according to the first aspect, wherein the tissue anchor has a longitudinal center axis and is configured to define a passage extending through the tissue anchor along the longitudinal central axis, and the helical tissue coupling element defines a portion of the passage of the tissue anchor; and an anchor deployment tool including a catheter having a catheter distal end; and a rotation driving body having a proximal end, a distal end, and a longitudinal through hole extending from the proximal end to the distal end thereof, wherein the proximal end of the rotation driving body is connected to the catheter distal end; and an elongate guide configured to be able to extend through the catheter and the longitudinal through hole of the rotation driving body, and to be detachably connected to the rotatable connection mechanism; wherein the rotation driving body is configured to guide the tissue anchor by extending within the passage of the helical tissue coupling element during anchoring of the tissue anchor to annulus tissue by rotating the tissue anchor.

The present disclosure will be described in more detail below in conjunction with the accompanying drawings.

### BRIEF DESCRIPTION OF THE DRAWINGS

The features and advantages of the above aspects of the disclosure will be more clearly understood from the following detailed description of exemplary embodiments given in conjunction with the accompanying drawings, which illustrate exemplary embodiments of the disclosure by way of non-limiting example, among which:

FIG. 1 is a schematic diagram of an annuloplasty apparatus according to an exemplary embodiment of the present disclosure;

FIG. 2A is a schematic diagram of an annuloplasty apparatus according to another exemplary embodiment of the present disclosure;

FIG. 2B is a schematic diagram of the annuloplasty apparatus of FIG. 2A anchored to the annulus tissue;

FIG. 3 is a perspective view of a tissue anchor used in the annuloplasty apparatus according to the present disclosure;

FIG. 4A is a perspective view of one example of a bar member of the annuloplasty apparatus of FIG. 1 or 2A, with a rotatable connection mechanism shown in partial cross-section;

FIG. 4B is a perspective view illustrating a plate-shaped body of the bar member shown in FIG. 4A;

FIG. 4C is a perspective view of a rotatable portion of the rotatable connection mechanism shown in FIG. 4A;

FIGS. 4D and 4E are exploded views of the rotatable portion of the rotatable connection mechanism shown in FIG. 4A;

FIGS. 5A to 5M show different embodiments of the rotatable connection mechanism;

FIG. 6A is a perspective structural diagram of a procedural apparatus for performing an annuloplasty;

FIGS. 6B and 6C show an anchor deployment tool of the procedural apparatus shown in FIG. 6A, wherein FIG. 6B shows a cross section of the rotation driving body of the anchor deployment tool in a partial cross-sectional view;

FIGS. 7A and 7B are schematic perspective views of delivering the anchor, wherein FIG. 7A shows that the head portion is at the proximal end of the rotation driving body of the anchor deployment tool, and FIG. 7B shows that the head portion is at the distal end of the rotation driving body of the anchor deployment tool;

FIG. 8A illustrates an initial rotational position of the anchor;

FIGS. 8B and 8C are a schematic view and a sectional view, respectively, showing that the distal tabs of the rotation driving body come into contact with the crossbar of the rotatable connection mechanism;

FIG. 8D is a schematic diagram showing that the distal tabs of the rotation driving body come into contact with the crossbar of the rotatable connection mechanism and that the rotation driving body continues to drive the anchor to rotate;

FIG. 9A is a schematic view showing that the proximal end of the helical tissue coupling element of the anchor begins to contact with the crossbar of the rotatable connection mechanism, wherein the head portion and the rotatable connection mechanism are shown in an enlarged view;

FIG. 9B is a schematic view showing that the rotation driving body continues to drive the anchor to rotate after the proximal end of the helical tissue coupling element of the anchor begins to contact with the crossbar of the rotatable connection mechanism, wherein the head portion and the rotatable connection mechanism are shown in an enlarged view;

FIG. 9C is a schematic view illustrating that the anchor is rotated to a final, proper position, wherein the gap between the plate-shaped implant and the annulus tissue has been eliminated or reduced, wherein the head portion and the rotatable connection mechanism are shown in an enlarged view;

FIG. 10A is a cross-sectional view showing that the anchor is rotated to a final, proper position;

FIG. 10B is a cross-sectional view showing that the guide is removed after the anchor has been rotated to the proper position;

FIG. 10C is a cross-sectional view showing that the distal tabs of the rotation driving body of the anchor deployment tool are about to be separated from the anchor; and

FIG. 10D is a schematic perspective view showing that the distal tabs of the rotation driving body of the anchor deployment tool have been separated from the anchor.

### DETAILED DESCRIPTION OF THE EMBODIMENTS

Exemplary embodiments of the present disclosure are described in detail below with reference to the accompanying drawings. It should be understood that embodiments having other arrangements may be employed without departing from the scope of the present disclosure. Accordingly, the following detailed description should not be construed as limiting the scope of the present disclosure. The scope of the present disclosure is defined by the appended claims and their equivalents. In all the drawings, the same reference numerals denote elements having the same or similar functions.

The annuloplasty apparatus is used for reshaping the mitral annulus and/or left ventricle of a subject so that the anterior and posterior leaflets are properly closed to prevent regurgitation by eliminating mitral insufficiency. The annuloplasty apparatus is typically secured to the annulus tissue by a tissue anchor and reduces the circumference of the mitral annulus by reducing the circumference of the annuloplasty apparatus so that the anterior and posterior leaflets are brought closer to prevent regurgitation. The annuloplasty apparatus may also be applied to the tricuspid valve for shaping and repair thereof.

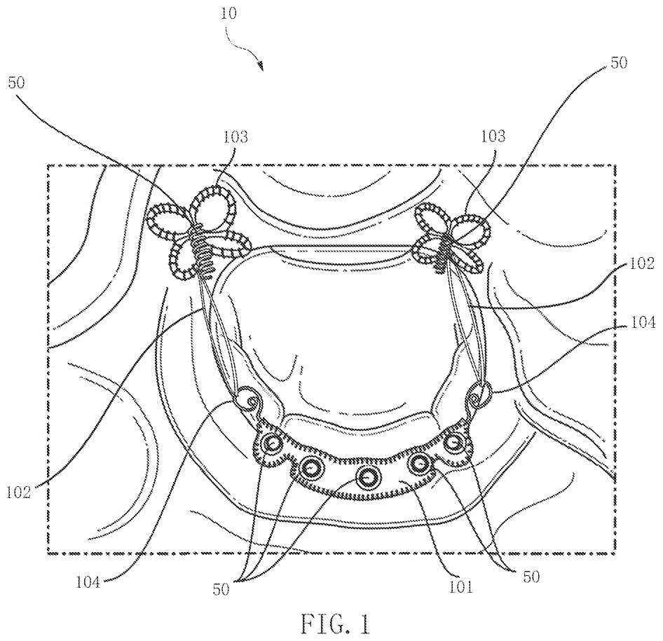

FIG. 1 shows a segmented annuloplasty apparatus 10 according to one embodiment of the present disclosure. The annuloplasty apparatus 10 comprises a bar member 101, other two pads 103, anchors 50 for anchoring the bar members 101 103, and a tensile member 102 that are deployed into the body through catheters, respectively. The bar member 101 and the pads 103 are independent members and are each secured to the annulus tissue by at least one tissue anchor 50. The bar member 101 has an elongated flat structure that can be bent slightly to match the anatomical structure of the mitral annulus at the location where the bar member 101 is anchored. The ends of the bar member 101 are provided with a loop feature 104. The pads 103 has a butterfly-shaped flat structure, and includes four petal portions extending radially outward from a central portion thereof. The number of the petal portions may be more than four or less than four, or no petal portion is provided, but is generally in the form of a butterfly-shaped overall flat structure. The top end of the bar member 103 is provided with a loop feature (not shown). For example, two tensile members 102 interconnect the bar member 101 with each of the pads 103 by respective loop features.

Preferably, the outer surfaces of the bar member 101 and the pads 103 are covered with polyethylene terephthalate (PET) to facilitate tissue ingrowth.

In implanting the annuloplasty apparatus 10, a catheter (not shown) is first introduced into the left atrium of the heart. The bar member 101 is then deployed into the left atrium by the catheter and is anchored to the posterior side of the mitral annulus in the left atrium by the anchor 50. The pads 103 are then deployed through the catheter and are anchored to the anterior side of the mitral annulus in the left atrium by the anchor 50. Flexible tensile members 102 are then deployed through the catheter and is attached to the respective loop features of both the bar member 101 and the pads 103, and tensions are then applied to the tensile members 102 to pull the bar member 101 and the pads 103 toward each other, thereby bringing the posterior and anterior sides of the mitral annulus closer.

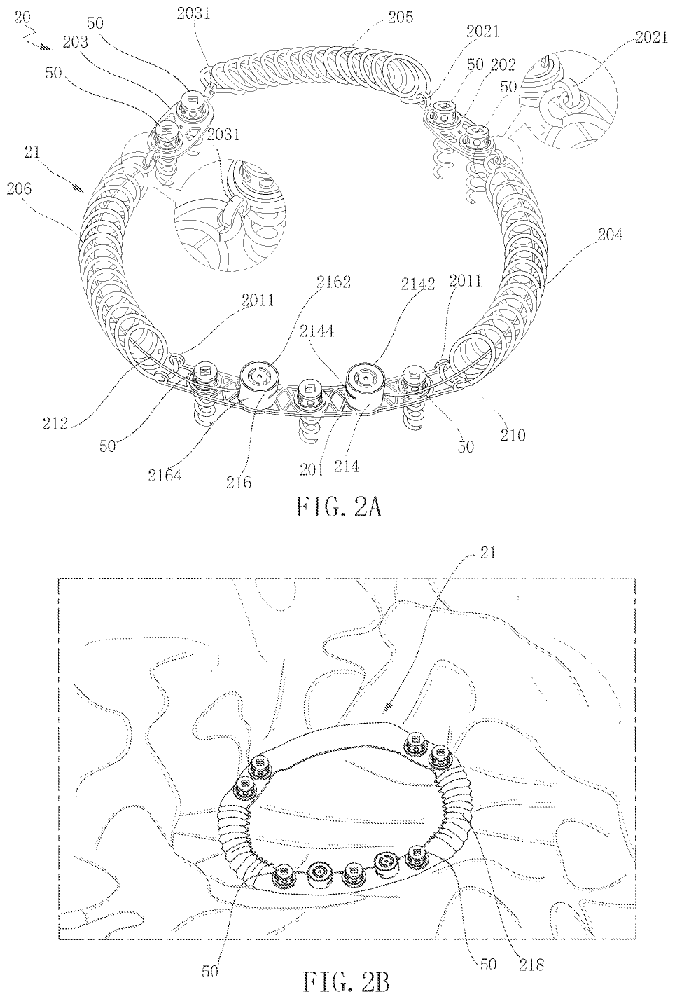

FIG. 2A illustrates an integral annuloplasty apparatus 20 according to another embodiment of the present disclosure. As shown in FIG. 2A, the annuloplasty apparatus 20 includes a shaping ring 21 and a plurality of anchors 50. The shaping ring 21 is a complete ring comprising bar members 201, 202, 203, extendable and compressible elements 204, 206 and an optional interconnecting element 205. The bar members 201, 202, 203 can be connected to one another via respective extendable and compressible elements 204, 206. The bar members 202, 203 may also be further connected by an interconnecting element 205 so that the shaping ring 21 forms a closed substantially annular shape. The extendable and compressible elements 204, 206 are connected to the respective bar members 201, 202, 203 by the respective free ends hooking to the respective loops 2011, 2021 and 2031 of the bar members 11, 12, 13. The interconnecting element 205 may also interconnect the bar members 202, 203 by hooking its respective free ends onto the respective loops 2021 and 2031 of the bar members 202, 203. Alternatively or additionally, the free ends of the extendable and compressible elements or the optional interconnecting element may be further welded to the respective loops.

The shaping ring 21 further comprises two adjustment wires 210, 212 and two cinching devices 214, 216. The cinching devices 214, 216 are respectively provided with housings 2144, 2164 fixed to the bar member 201 and with reels 2142, 2162 respectively rotatably arranged in the housings 2144 and 2164. One end of the adjustment wire 210 is connected to the loop 2021 of the bar member 202, and the other end thereof extends through a threading hole (not shown) provided in the housing 2144 to be connected to the reel 2142 of the cinching device 214. One end of the adjustment wire 212 is connected to the loop 2031 of the bar member 203, and the other end thereof extends through a threading hole (not shown) provided in the housing 2162 to be connected to the reel 2162 of the cinching device 216.

The reel 2142 may be driven to rotate in a tightening direction and a release direction. When the reel 2142 of the cinching device 214 is driven to rotate in the tightening direction, the adjustment wire 210 is wound around the reel 2142 with the rotation of the reel 2142 so as to shorten the length of the adjustment wire 210, thereby pulling the bar members 201 and 202 toward each other. Similarly, when the reel 2162 of the cinching device 216 is rotated, the length of the adjustment wire 212 can also be shortened, thereby pulling the bar members 201 and 203 toward each other. Thus, the annular surface of the annular shaping ring 21 is reduced.

Because the bar members 201, 202, 203 are connected to the physiological valve annulus by means of tissue anchors, the physiological valve annulus contracts as the annulus of the shaping ring 21 contracts, thereby reducing the circumference of the physiological valve annulus and bringing the anterior and posterior leaflets closer to each other. In this way, the regurgitation caused by insufficient closure of the anterior and posterior leaflets can be eliminated.

The adjustment wire 210 extends within the interior cavity defined by the extendable and compressible element 204, and the adjustment wire 212 extends within the interior cavity defined by the extendable and compressible element 206. Alternatively, the adjustment wires 210, 212 may extend at least partially within the interior cavity defined by the respective extendable and compressible elements. For example, in the case where the extendable and compressible element is a coil element, the adjustment wires 210, 212 may alternately pass through the coils of the respective coil elements. The advantage of this arrangement is that the coil element can better snugly abut against the annulus tissue. Alternatively, the adjustment wires 210, 212 may also extend outside the respective extendable and compressible elements.

The interconnecting element 205 may comprise a linear element, such as a wire, composed of a shape memory material. Preferably, the interconnecting element 205 is constituted by an inclined flat-shaped coil element, the coils of which can be configured to be inclined with respect to the longitudinal centre line of the interconnecting element 205, thereby enabling the interconnecting element 205 to snugly abut against the surface of the annulus tissue. Preferably, the extendable and compressible elements 204, 206 are constituted by inclined flat-shaped coil elements.

As shown in FIG. 2B, the shaping ring 21 covered with the fabrics 218 is secured to the annulus tissue by the anchor 50, the cinching devices 214, 216 are adjusted until an appropriate tension of the adjustment wires 210, 212 and/or approaching of the annulus tissues is achieved.

In the annuloplasty apparatuses 10, 20 shown in FIGS. 1 and 2A which are formed by connecting the bar members and pads by a contractile bridging element (the tensile member 102 or the extendable and compressible elements 204, 206), the bar members 101, 201 to 203 and the pads 103 are anchored to the annulus tissue by the tissue anchors 50 shown in FIG. 3. Further, the number of anchor 50 used in FIGS. 1 and 2A is merely exemplary, and other suitable numbers of the anchor 50 may be used to anchor the bar members 101, 201 to 203 and the pads 103. In the following description, the term “plate-shaped implant” will be used to exemplarily represent the bar members 101, 201, 202 and/or 203 and the pads 103 described above.

As shown in FIG. 3, the anchor 50 may be a helical tissue anchor and include a head portion 502, and a helical tissue coupling element 504 having a proximal end secured to the head portion 502, and a distal end being a sharp tip, in order to penetrate into the annulus tissue. A non-circular engagement opening 506, which extends through the head portion 502 along the longitudinal central axis of the anchor 50, is centrally disposed in the proximal end face of the head portion 502. The non-circular engagement opening 506 may be a polygonal hole, such as triangular, quadrangular, pentagonal, hexagonal, and the like. Preferably, the non-circular engagement opening 506 is quadrilateral. The non-circular engagement opening of the head portion 502 is used to drivingly engage a rotation driving body 6024 having a mating cross-sectional shape described below, to drive the anchor 50 into the annulus tissue by rotation. The rotation driving body 6024 will be described in detail later.

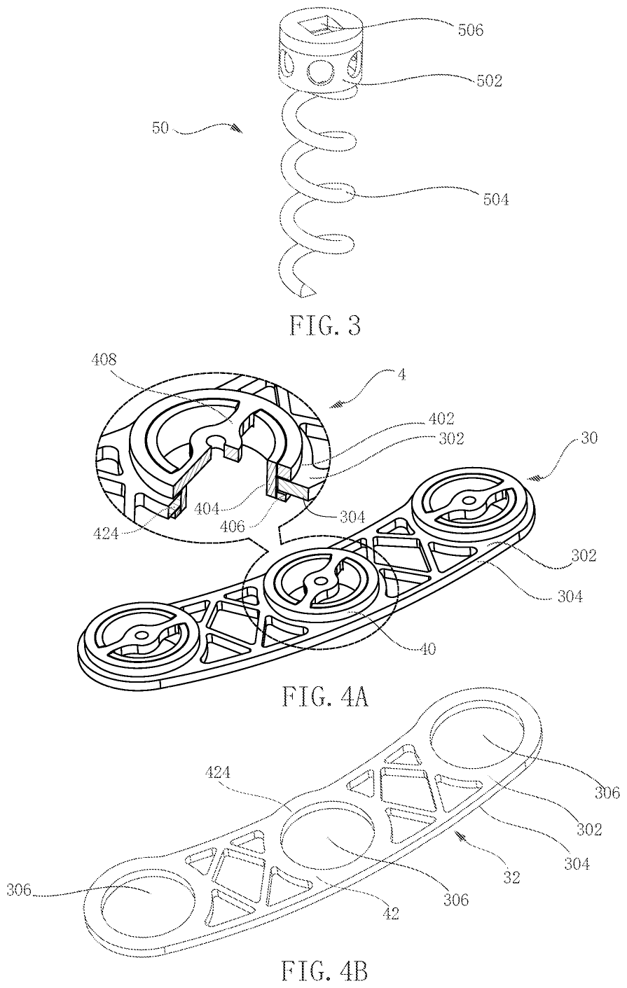

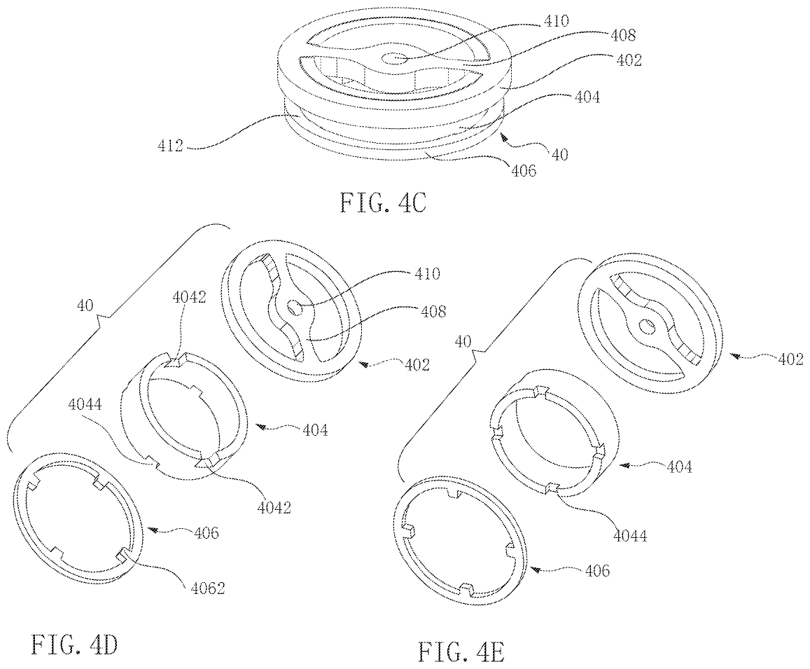

FIG. 4A shows a perspective view representing a plate-shaped implant 30, in which the rotatable connection mechanism 4 is shown in a partial cross-sectional view. FIG. 4B shows a perspective view of the plate-shaped body 32 of the plate-shaped implant 30, FIG. 4C shows the perspective view of a rotatable portion 40 in the rotatable connection mechanism 4, and FIGS. 4D and 4E show an exploded perspective view of the rotatable portion 40.

As shown in FIGS. 4A and 4B, the plate-shaped implant 30 may be an elongated flat structure, and includes a first surface 302, a second surface 304 opposite to the first surface 302, and three circular through holes 306 extending from the first surface 302 to the second surface 304. The specific shape of the elongated flat structure of the plate-shaped implant 30 and the number of the circular through holes 306 can be appropriately adjusted according to the anatomical characteristics of the desired annulus tissue to be implanted. For example, for the bar member 101 shown in FIG. 1, five circular through holes 306 may be provided; and for the bar members 202, 203 shown in FIG. 2A, only two circular through holes 306 may be provided; and for the bar member 201 shown in FIG. 2A, three circular through holes 306 may be provided. Further, the spacing between the circular through holes can be appropriately adjusted according to actual needs.

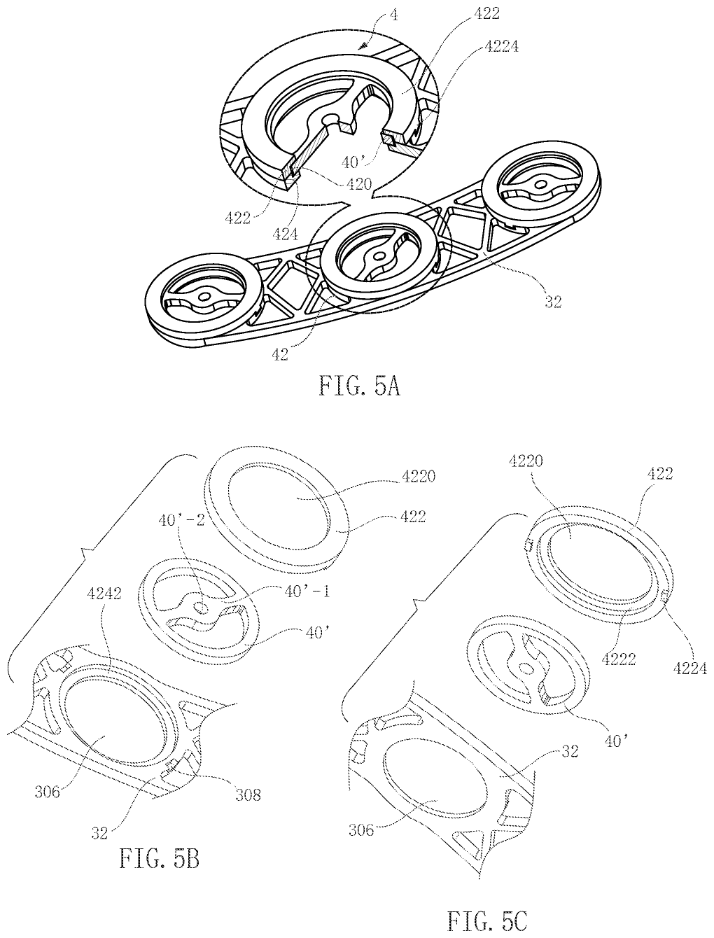

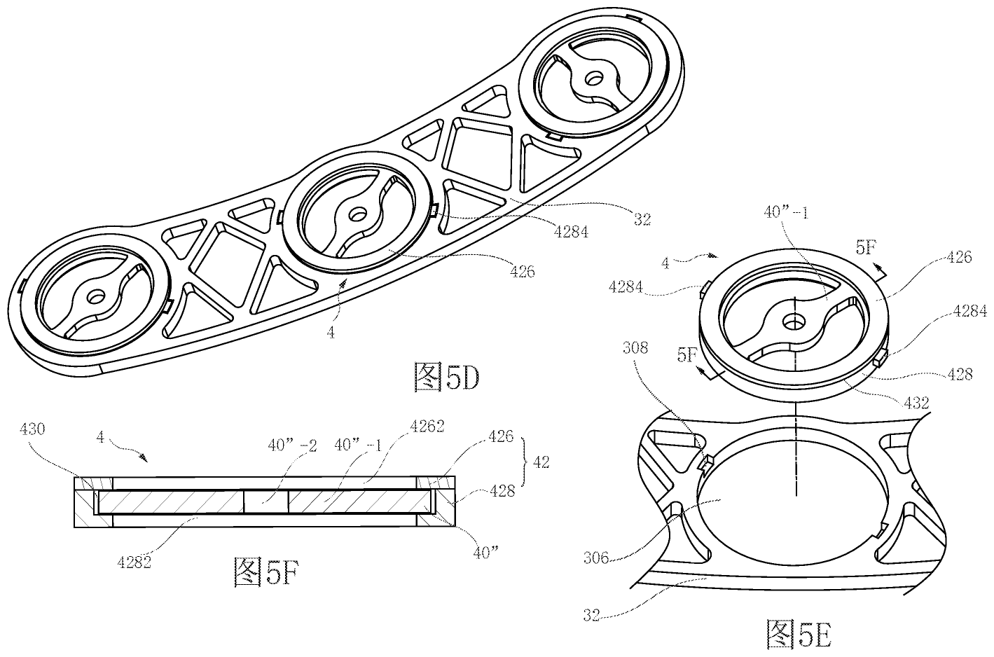

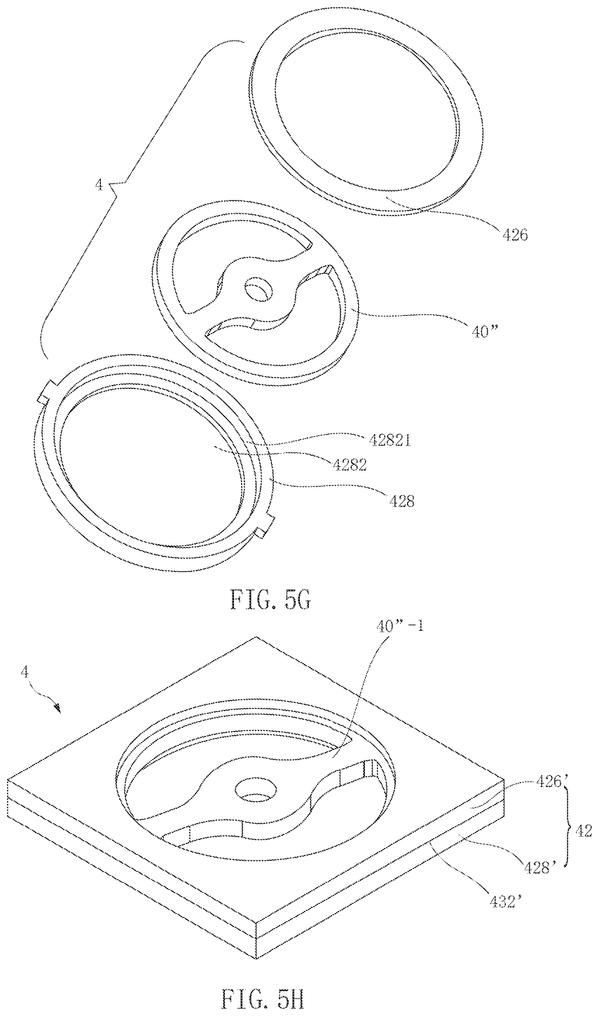

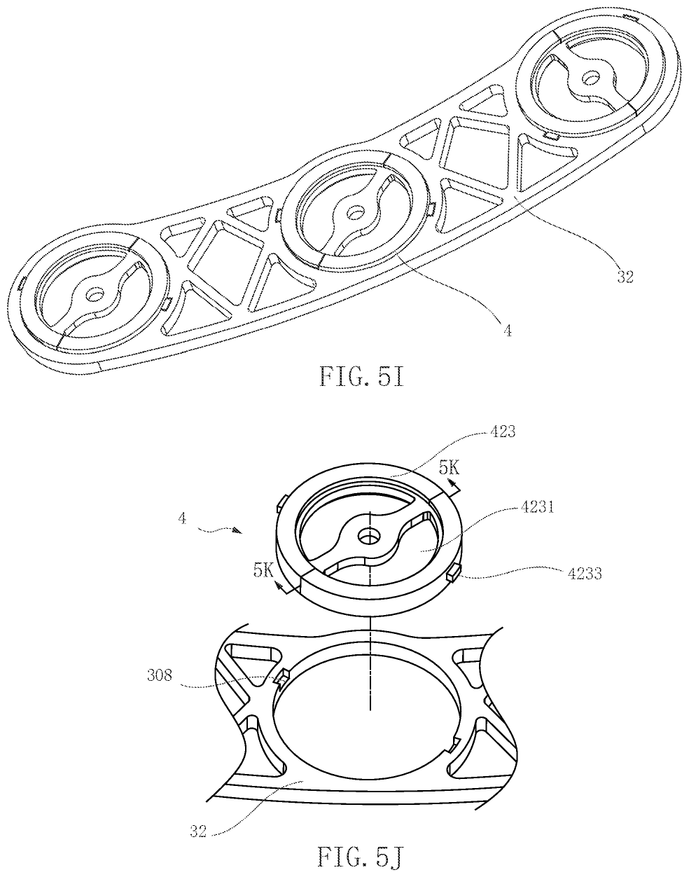

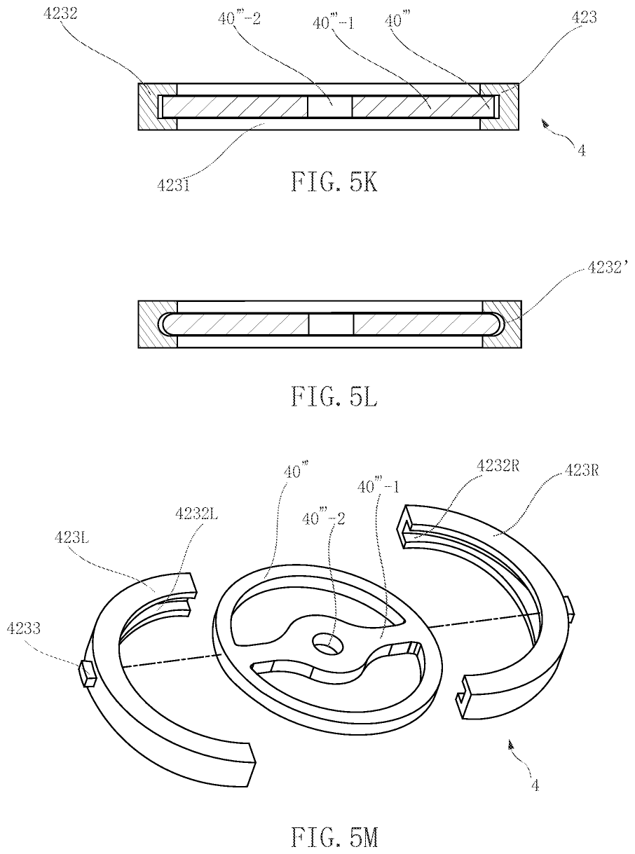

Continuing with reference to FIG. 4A and FIGS. 5A to 5M, the plate-shaped implant 30 may be provide with a rotatable connection mechanism 4 comprising a fixed portion 42 and a rotatable portion 40. The fixed portion 42 is fixedly provided to the plate-shaped body 32, and the rotatable portion 40 is configured to be rotatable with respect to the plate-shaped body 32. In this way, when the proximal end of the helical tissue coupling element 504 of the tissue anchor 50 is in contact with connection portions (e.g., the crossbars 408, 40′-1, 40″-1, 40′″-1) of the rotatable portion 40 of the rotatable connection mechanism 4, the tissue anchor 50 is allowed to rotate further relative to the plate-shaped implant 30 to eliminate or reduce the gap created between the plate-shaped implant 30 and the tissue.

In a preferred embodiment as shown in FIGS. 4A to 4E, the rotatable connection mechanism 4 may include a rotatable portion 40 and a fixed portion 42. The rotatable portion 40 may include a tubular body 404 having a proximal end and a distal end, which may be arranged in the circular through hole 306 of the plate-shaped implant 30. The fixed portion 42 may be constituted by a portion 424 of the plate-shaped body 32 defining the circular through hole 306 thereof. The outer diameter of the tubular body 404 may be equal to or slightly smaller than that of the circular through hole 306, so that the outer circumferential surface of the tubular body 404 can be slidably engaged with the inner circumferential surface of the circular through hole 306, thereby enabling the tubular body 404 to rotate in the circular through hole 306. As shown in FIG. 4A, the proximal and distal ends of the tubular body 404 may protrude from the first surface 302 and the second surface 304 of the plate-shaped implant 30 respectively, when the tubular body 404 is arranged within the circular through hole 306. The rotatable portion 40 may also include a top ring 402 and a bottom ring 406. The outer diameters of the top ring 402 and the bottom ring 406 may be larger than the inner diameter of the circular through hole 306, and the inner diameter may be equal to or smaller than the inner diameter of the circular through hole 306. The top ring 402 can be fixed to the proximal end of the tubular body 404 around the tubular body 404, the bottom ring 406 can be fixed to the distal end of the tubular body 404 around the tubular body 404, thus, an annular circumferential groove 412 extending in the circumferential direction of the tubular body 404 is defined between the top ring 402, the tubular body 404 and the bottom ring 406.

Alternatively, the top ring 402 and the bottom ring 406 may not be arranged around the tubular body 404. For example, the proximal and distal ends of the tubular body 404 may be substantially flush with the first surface 302 and the second surface 304 of the plate-shaped implant 30 when the tubular body 404 is arranged within the circular through hole 306. In this case, the inner diameters of the top ring 402 and the bottom ring 406 may be substantially the same or slightly larger than the inner diameter of the tubular body 404, but smaller than the outer diameter thereof. The outer diameters of the top ring 402 and the bottom ring 406 may be greater than the inner diameter of the circular through hole 306. In this way, the top ring 402 and the bottom ring 406 may be disposed substantially concentrically with the tubular body 404 at the proximal end face and the distal end face of the tubular body 404 respectively, so as to likewise define the annular circumferential groove 412 described above.

In assembling the top ring 402, the tubular body 404, and the bottom ring 406 to the plate-shaped body 32, the portion 424 of the plate-shaped body 32, which portion defines the circular through hole 306, may be received in the annular circumferential groove 412 defined by the top ring 402, the tubular body 404 and the bottom ring 406, so that the rotatable portion can be rotated with respect to the portion 424 and thus with respect to the plate-shaped body 32. The top ring 402, the tubular body 404 and the bottom ring 406 may be fixed together by welding so that they can rotate as a whole with respect to the plate-shaped implant 30.

In the embodiment shown in FIGS. 4D and 4E, the top ring 402, the tubular body 404, and the bottom ring 406 are separate elements from each other. However, one of the top ring 402 and the bottom ring 406 may be integral with the tubular body 404. For example, the top ring 402 may be integral with the tubular body 404, and the bottom ring 406 may be secured (e.g., welded) to the distal end of the tubular body 404 around the distal end face of the tubular body 404 after the integral tubular body and top ring 404 are assembled into the circular through hole 306, thereby defining an annular circumferential groove 412 of the rotatable portion 40 in which groove 412 the portion 424 is received.

The top ring 402 may include a connection portion, and the tissue anchor 50 may secure the plate-shaped implant 30 to the annulus tissue via the helical tissue coupling element 504 and the connection portion. In the example shown in FIGS. 4D and 4E, the connection portion may be a crossbar 408 fixed radially to the inner peripheral wall of the top ring 402. In this case, the crossbar 408 may be formed integrally with the top ring 402 or may be fixed to the inner peripheral wall of this top ring by welding.

It should be noted that examples of the connection portion are not limited to the crossbar 408 as shown, but may also take other forms or shapes, as long as the helical tissue coupling element 504 of the tissue anchor 50 is able to secure the plate-shaped implant 30 to the annulus tissue through this connection portion. For example, the connection portion may take the form of a fan-shaped suspending wall (not shown) extending toward the center of the top ring 402 inwardly from the inner peripheral surface of the top ring 402 and substantially parallel to the distal end face of the top ring 402. The fan-shaped suspending wall occupies a part of the inner circumferential surface of the top ring 402 in the circumferential direction of the inner peripheral surface of the upper ring 402, to define an opening between the fan-shaped suspending wall and the inner peripheral surface of the top ring 402, which opening allows passage of the helical tissue coupling element 504 of the helical tissue anchor 50. The fan-shaped suspending wall may be a top wall at the proximal end of the inner peripheral surface of the top ring 402, a bottom wall at the distal end thereof, or an intermediate wall between the proximal end and the distal end of the inner peripheral surface of the top ring 402.

Further, a threaded hole, which is used for detachably connecting with an external thread 6042 of the distal end of the elongate guide 604 described below, may be provided at a portion of the fan-shaped suspending wall, which portion is located at the center of the top ring 402. The fan-shaped suspending wall may occupy ⅙ to ⅝ or other proportions of the circumference of the inner peripheral surface of the top ring 402, as long as the opening between the fan-shaped suspending wall and the inner peripheral surface of the top ring 402 allows the helical tissue coupling element 504 to pass through.

Alternatively, the crossbar 408 may also be radially fixed into the proximal end face or the distal end face of the top ring 402. In this case, it is preferred that two notches (not shown), which are opposed radially and are used for receiving the crossbars 408, may be provided in the proximal end face or the distal end face of the top ring 402. It is preferred that, after the crossbars 408 are arranged in the two notches (not shown), the crossbars 408 are secured to the two notches by, for example, welding.

Preferably, the proximal end face of the tubular body 404 may be provided with two notches 4042 that are opposed radially and are configured to receive the crossbars 408. In this case, the distal end face of the tubular body 404 may be provided with a plurality of notches 4044 configured to receive a number of projections 4062, respectively, disposed radially inwardly on the inner peripheral surface of the bottom ring 406. FIGS. 4D and 4E show four projections 4062 and corresponding four notches 4044. However, other numbers of projections 4062 and corresponding numbers of notches 4044 may be provided, such as two, three, four, or more.

Further preferably, when the crossbar 408 is received in the notches 4042, the proximal end faces of the top ring 402 and the crossbar 408 are flush with the proximal end face of the tubular body 404, as shown in FIG. 4C.

Alternatively, the crossbar 408 may be radially secured to the inner peripheral wall of the tubular body 404 between the proximal end and the distal end of the tubular body 404. In this case, the top ring 402 may take the same structure as the bottom ring 406, that is, a plurality of projections (not shown) may be provided that project radially inwardly from the inner peripheral surface of the top ring 402. The proximal end of the tubular body 404 may take the same structure as the distal end, that is, a corresponding number of notches (not shown) for receiving the plurality of projections of the top ring 402 may be provided.

As shown in FIGS. 4D and 4E, the center of the crossbar 408 is provided with a guide engaging portion for being detachably connected with an elongate guide 604 described below. Preferably, the guide engaging portion is a threaded hole 410 that is detachably engaged with an external thread 6042 disposed at the distal end of the guide 604.

In the embodiment of the rotatable connection mechanism 4 described above, the rotatable portion 40 of the rotatable connection mechanism 4 consists of three discrete elements, namely a top ring 402, a tubular body 404 and a bottom ring 406, whereas the fixed portion 42 of the rotatable connection mechanism 4 is constituted by a portion 424 of the plate-shaped body 32 defining the circular through hole 306, wherein the portion 424 is received in the annular circumferential groove 412 defined by the top ring 402, the tubular body 404 and the bottom ring 406, in order to enable the rotatable portion 40 to rotate relative to the fixed portion 42 (in the embodiment, the portion 424) and thus relative to the plate-shaped implant 30.

In an alternative embodiment, as shown in FIGS. 5A to 5C, the rotatable portion 40 of the rotatable connection mechanism 4 may be constituted by a circular ring 40′, which is similar to the top ring 402 shown in FIG. 4D. Specifically, the circular ring 40′ may include a connection portion, and the tissue anchor 50 may secure the plate-shaped implant 30 to the annulus tissue via the helical tissue coupling element 504 and the connection portion. In the example shown in FIGS. 5A to 5C, the connection portion may be a crossbar 40′-1 fixed radially to the inner peripheral wall of the circular ring 40′. In this case, the crossbar 40′-1 may be formed integrally with the circular ring 40′ or may be fixed to the inner peripheral wall of the circular ring by welding.

Alternatively, the crossbar 40′-1 may also be radially fixed into the proximal end face or the distal end face of the circular ring 40′. In this case, it is preferred that two notches (not shown), which are opposed radially and are used for receiving the crossbars 40′-1, are provided in the proximal end face or the distal end face of the circular ring 40′. It is more preferred that, after the crossbars 40′-1 are arranged in the two notches (not shown), the crossbars 40′-1 are fixed to the two notches, for example, by welding. Similarly, the crossbar 40′-1 is centrally provided with a guide engaging portion, such as a threaded hole 40′-2, for being detachably connected with the elongate guide 604. In addition, the crossbar 40′-1 may also be replaced by the fan-shaped suspending wall (not shown) as described above.

Further referring to FIGS. 5A to 5C, the fixed portion 42 of the rotatable connection mechanism 4 may include: a portion 424 of the plate-shaped body 32 defining the circular through hole 306; and a top plate 422 having a circular through hole 4220, wherein the top plate 422 may be fixedly (e.g., by welding) superimposed on the portion 424 with the circular through holes 306 and 4220 being substantially concentric. An L-shaped first circumferential groove 4222 extending in the circumferential direction is provided in the inner peripheral wall of the circular through hole 4220 of the top plate 422, and an L-shaped second circumferential groove 4242 extending in the circumferential direction is provided in the inner peripheral wall of the circular through hole 306 of the plate-shaped body 32. The first and second circumferential grooves 4222 and 4242 together define an annular circumferential groove 420. A circular ring 40′ constituting the rotatable portion 40 may be slidably received in the annular circumferential groove 420 so as to enable the circular ring 40′ to rotate relative to the fixed portion 42 and thus to the plate-shaped implant 30.

Preferably, as shown in FIG. 5C, the top plate 422 may be provided with at least one projection 4224 (two are shown in FIG. 5C) on the end face facing the plate-shaped body 32. The protrusions 4224 may be received in corresponding notches 308 provided in the plate-shaped body 32. The top plate 422 may be fixed to the plate-shaped body 32 by welding after the protrusions 4224 are received in the corresponding notches 308.

In the embodiment shown in FIGS. 5A to 5C, the annular circumferential groove 420 is constituted by an L-shaped first circumferential groove 4222 and an L-shaped second circumferential grooves 4242. However, the annular circumferential groove 420 may be constituted only by an L-shaped circumferential groove (not shown) provided in the top plate 422 or the portion 424. In addition, FIGS. 5A to 5C show a top plate 422 whose external shape is circular. However, the present disclosure is not limited thereto, and the top plate 422 may have other external shapes, for example, a square shape.

FIGS. 5D to 5G show a further embodiment of the rotatable connection mechanism 4. In this embodiment, the rotatable portion 40 of the rotatable connection mechanism 4 is constituted by the circular ring 40″, which is similar to the top ring 402 shown in FIG. 4D. Specifically, the circular ring 40″ may include a connection portion, and the tissue anchor 50 may secure the plate-shaped implant 30 to the annulus tissue via the helical tissue coupling element 504 and the connection portion. In the example shown in FIGS. 5D to 5G, the connection portion may be a crossbar 40″-1 fixed radially to the inner peripheral wall of the circular ring 40″. In this case, the crossbar 40″-1 may be formed integrally with the circular ring 40″ or may be fixed to the inner peripheral wall of the circular ring by welding. In addition, the crossbar 40″-1 may also be replaced by the fan-shaped suspending wall (not shown) as described above.

Alternatively, the crossbar 40″-1 may also be radially fixed into the proximal end face or the distal end face of the circular ring 40″. In this case, it is preferred that two notches (not shown), which are opposed radially and are used for receiving the crossbars 40″-1, are provided in the proximal end face or the distal end face of the circular ring 40″. It is more preferred that, after the crossbars 40″-1 are arranged in the two notches (not shown), the crossbars 40″-1 are fixed to the two notches, for example, by welding. Similarly, the crossbar 40″-1 is centrally provided with a guide engaging portion, such as a threaded hole 40″-2, for being releasably connected with the elongate guide 604.

The fixed portion 42 of the rotatable connection mechanism 4 may be at least partially received in the circular through hole 306 of the plate-shaped body 32 and may be fixed to the plate-shaped body 32, for example, by welding. In this embodiment, the fixed portion 42 may include a top plate 426 having a circular through hole 4262, and a circular base 428 having a stepped circular through hole 4282, wherein the large diameter hole 42821 of the stepped circular through hole 4282 and the top plate 426 define a substantially circumferential groove 430. The circular ring 40″ may be received in the circumferential groove 430 so as to rotate relative to the fixed portion 42 and thus to the plate-shaped implant 30.

Preferably, the outer peripheral wall of the circular base 428 is provided with at least one projection 4284 which can be received in a corresponding notch 308 provided in the inner peripheral wall in the circular through hole 306 of the plate-shaped body 32.

Referring to FIGS. 5F and 5G, the fixed portion 42 is a split-type fixed base including a top plate 426 and a base 428, and FIG. 5E shows a boundary line between the top plate 428 and the base 428 at reference numeral 432. The top plate 426 is secured to the base 428, such as by welding, after the circular ring 40″ is placed in the circumferential groove 430, and the fixed portion 42 composed of the top plate 426 and the base 428 is then placed in the through hole 306 and is further fixed to the plate-shaped body 32, for example, by welding.

FIGS. 5E, 5G show the fixed portion 42 (i.e., the top plate 426 and the base 428) having a circular shape, but the present disclosure is not limited thereto. For example, FIG. 5H illustrates the fixed portion 42 having other shapes, such as a square shape. Accordingly, the plate-shaped body 32 is provided with a through hole (not shown) having a shape corresponding to the other shapes of the fixed portion 42. The reference numeral 432′ shows a boundary line 432′ between the top plate 426′ and the base 428′. The other structures of the top plate 426′ and the base 428′ are the same as those of the top plate 426 and base plate 428 except for the external shape.

FIGS. 5I to 5M show a rotatable connection mechanism 4 in another embodiment. The rotatable connection mechanism 4 of this embodiment is different from the one shown in FIGS. 5D to 5G only in the fixing seat 423 constituting the fixed portion 42. The circular ring 40′″ (constituting the rotatable portion 40) and its connection portion are the same as the circular ring 40″ and its connection portion. For example, the connection portion of the circular ring 40′″ may comprise a crossbar 40′″-1 radially fixed to the inner peripheral wall of the circular ring 40″. The center of the crossbar 40′″-1 may be provided with a guide engaging portion, such as a threaded hole 40′″-2, for being detachably connected with the elongate guide 604. In addition, the crossbar 40′″-1 may also be replaced by the fan-shaped suspending wall (not shown) as described above. As shown in FIG. 5K, the fixing seat 423 includes a circular through hole 4231, the inner peripheral wall of which is provided with an annular circumferential groove 4232 extending in the circumferential direction. The circular ring 40′″ is slidably received in the circumferential groove 4232, so as to be rotatable relative to that fixed seat 432.

As shown in FIG. 5M, the fixed seat 423 may include a left fixed seat half 423L having a left circumferential groove 4232L and a right fixed seat half 423R having a right circumferential groove 4232R. When the left fixed seat half 423L and the right fixed seat half 423R are combined with each other to constitute the split-type fixed base 423, the left circumferential groove 4232L and the right circumferential groove 4232R constitute a complete annular circumferential groove 4232. It is to be noted that the left fixed seat half 423L and the right fixed seat half 423R are combined with each other and fixed to each other, for example, by welding, after the circular ring 40′″ is received in the left circumferential groove 4232L and the right circumferential groove 4232R.

FIG. 5K shows a U-shaped circumferential groove 4232. Alternatively, as shown in FIG. 5L, the circumferential groove 4232 may be a circumferential groove 4232′ having a semicircular cross section. Accordingly, the circular ring 40′″ also has a semicircular cross section to be slidably received in the circumferential groove 4232′.

Preferably, the outer peripheral wall of the left fixed seat half 423L and/or the right fixed seat half 423R is provided with at least one projection 4233. The at least one projection 4233 may be received in a corresponding notch 308 (see FIG. 5J) on the inner peripheral wall of the circular through hole 306 of the plate-shaped body 32.

FIG. 5M shows a split-type fixed base 423 consisting of two semi-circular fixed seat halves 423L and 423R, the external shape of which is circular. However, the split-type fixed base 423 may have other external shapes (e.g., square), and accordingly, the plate-shaped body 32 may have a through hole 306 whose shape is corresponding to other external shapes (e.g., square) of the split-type fixed base 423.

In the embodiments shown in FIGS. 5D to 5F and 5I to 5J, the distal end surface of the base 428 and/or the fixed seat 423 is preferably flush with the second surface 304 of the plate-shaped body 32 when the base 428 and/or the fixed seat 423 is mounted into the through hole 306 of the plate-shaped body 32, so that the second surface 304 of the plate-shaped implant 30 is brought into better conformity with the annulus tissue, thereby reducing the gap that exists between the two.

In the above example, it is described that the rotatable connection mechanism includes both the rotatable portion and the fixed portion. However, this is merely for ease of illustration of the present disclosure and is not intended to be limiting. For example, the rotatable connection mechanism may comprise only the rotatable portion as described above. In this case, a bar member (e.g., the plate-shaped body 32) may be additionally provided with the fixed portion as described above.

Description is continued below with reference to FIG. 3. The tissue anchor 50 as shown in FIG. 3 may secure the plate-shaped implant 30 to the annulus tissue by the connection portion (such as crossbars 408, 40′-1, 40″-1, 40′″-1) of the rotatable connection mechanism 4 provided on the plate-shaped implant 30. In an end procedure of drilling the anchor 50 into tissue by torque, the proximal end of the helical tissue coupling element 504 of the tissue anchoring element 50 abuts against the connection portion. If the connection portion is non-rotatably and fixedly disposed directly in the circular through hole 306 of the plate-shaped body 32, the tissue anchor 50 cannot be rotated further after the proximal end of the helical tissue coupling element 504 is in contact with the connection portion, which may create a gap between the implant 30 and the annulus tissue, which in turn results in displacement or even detachment of the tissue anchor 50. In the plate-shaped implant 30 of the present disclosure, since it is provided with the rotatable connection mechanism 4, after the proximal end of the helical tissue coupling element 504 of the tissue anchor 50 abuts against the connection portion, it is still possible to continue to drive the anchor 50 to rotate, so that the helical tissue coupling element 504 continues to rotate and advances into the annulus tissue to eliminate or reduce the gap between the plate-shaped implant 30 and annulus tissue, thereby making the anchoring more secure.

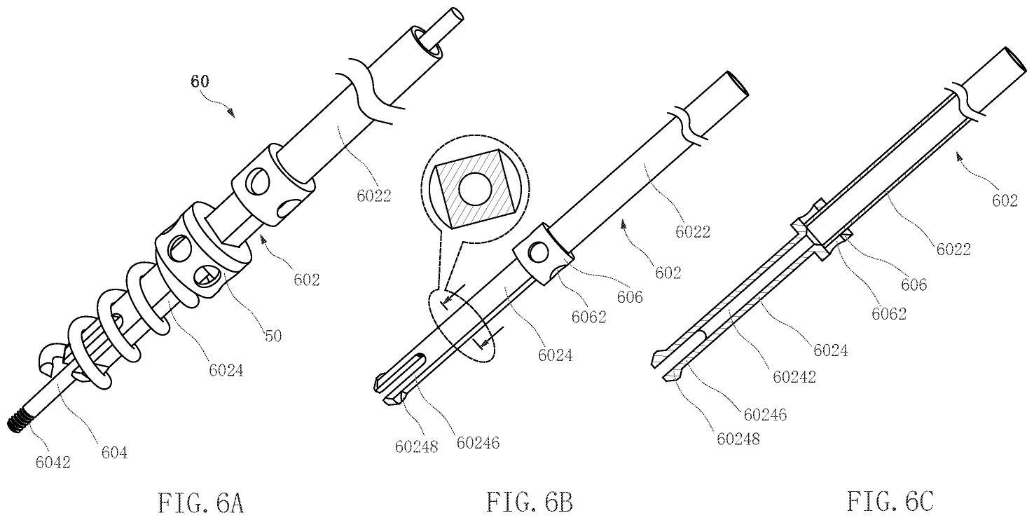

A procedural apparatus for driving the tissue anchor 50 to secure the plate-shaped implant 30 to the annulus tissue is described below with reference to FIGS. 6A, 6B, and 6C. It should be noted that in the following description, the rotatable connection mechanism 4 shown in FIGS. 4A to 4E is taken as an example to explain how the plate-shaped implant 30 is anchored to the annulus tissue. However, those skilled in the art will recognize that the rotatable connection mechanism shown in FIGS. 5A to 5M is also capable of performing the same functions and obtaining the same effects.

FIG. 6A shows a procedural apparatus 60 used to perform an annuloplasty, and FIGS. 6B and 6C show an anchor deployment tool 602 of the procedural apparatus 60.

As shown in FIG. 6A, the procedural apparatus 60 may include a tissue anchor 50, an anchor deployment tool 602, and an elongate guide 604, as shown in FIG. 3. The tissue anchor 50 has a longitudinal central axis (not shown) and defines a passage extending through the tissue anchor along the longitudinal center axis, wherein the head portion 502 and the helical tissue coupling element 504 together define the passage. The anchor deployment tool 602 may include a catheter 6022 having a proximal end and a distal end, and a rotation driving body 6024 having a proximal end and a distal end and a longitudinal through hole 60242 extending from the proximal end to the distal end. The proximal end of the rotation driving body 6024 is connected to the distal end of catheter 6022.

The elongate guide 604 may extend through the longitudinal through hole 60242 and the rotation driving body 6024 of the catheter 6022 and has external thread 6042 at the distal end. The external thread 6042 is for being detachably engaged with a threaded hole 410 provided at the central portion of the crossbar 408 of the rotatable portion 40.

The rotation driving body 6024 is elongated and the shapes of all cross sections along their longitudinal length match the shape of the non-circular engagement opening 506 of the head portion 502. In this way, when the tissue anchor 50 sleeves on the rotation driving body 6024, the rotation driving body 6024 is able to transfer torque to the anchor 50 during rotation, i.e., drive the anchor 50 to rotate. Since the shapes of all cross sections along the longitudinal length of the rotation driving body 6024 match the shape of the non-circular engagement opening 506 of the head portion 502, the rotation driving body 6024 is therefore always able to drive the anchor 50 to rotate by transferring torque to the anchor 50 through rotational movement while the anchor 50 is sliding in the distal direction along the rotation driving body 6025, thereby driving the anchor 50 into the annulus tissue.

The above-described longitudinal through hole 60242 is provided in the rotation driving body 6024 along the longitudinal direction. Preferably, the distal end of the rotation driving body 6024 includes a furcation body 60246. In the example shown in FIGS. 6B and 6C, the furcation body 60246 includes two legs, but may also include three or more legs.

The distal end portion of the furcation body 60246 may be provided with tabs 60248 that project radially outward. For example, the tabs 60248 may be provided radially outward on the distal end of one or more of the legs of the furcation body 60246. The furcation body 60246 may be made of a shape memory material such as Nitinol. The natural state of the legs or tabs 60248 may be set to separate naturally but have a tendency to converge inwardly. The elongate guide 604 may be inserted between the tabs 60248 such that the tabs 60248 remain in the separated state. When the elongate guide 604 is removed from between the tabs 60248, the tabs 60248 may approach each other inwards under force. When the external force is removed, the tabs 60248 may remain in a naturally separated state. Preferably, the naturally separated state of the tabs 60248 may also be arranged such that the tabs 60248 are bent inwards towards each other. In some embodiments, the elongate guide 604 may be inserted between the tabs 60248 such that the tabs 60248 may be pushed outwardly with abutting against the elongate guide 606, thereby preventing the tabs 60248 from passing through non-circular engagement opening 506 of the head portion 502 of the tissue anchor 50. When the elongate guide 604 between the tabs 60248 is removed, the tabs 60248 approach one another inwardly, thereby allowing the tabs 60248 to pass through the non-circular engagement opening 506 of the head portion 502 of the tissue anchor 50. The following description is made with the separated state of the tabs 60248 set to a naturally separated state.

As shown in FIGS. 6B and 6C, the anchor deployment tool 602 also includes a catheter connection portion 606 at the proximal end of the rotation driving body 6024. The catheter connection portion 606 connects the proximal end of the rotation driving body 6024 and the distal end of the catheter 6022 as one piece and may define a longitudinal passage (not labeled) that communicates the inner cavity of the catheter 6022 with the longitudinal through hole 60242 of the rotation driving body 6024. In the example shown in FIGS. 6B and 6C, the catheter connection portion 606 is a cylindrical element integral with the proximal end of the rotation driving body 6024, the distal end of the catheter 6022 is inserted into the cylindrical element and is welded to the catheter connection portion 606 by means of a plurality of process holes 6062 provided in the outer peripheral wall of the cylindrical element. The transversal dimension of the catheter connection portion 606 is preferably greater than the cross-sectional dimension (e.g., the length or width of the cross section) of the rotation driving body 6024 in order to be able to push the tissue anchor 50 towards a distal direction.

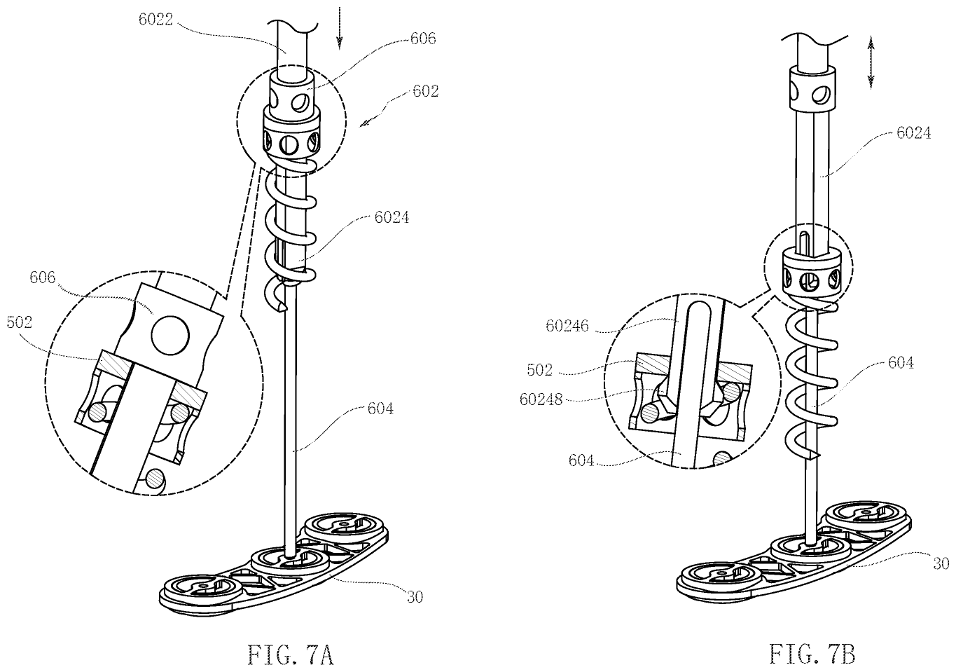

As shown in FIG. 7A, in the case where the tissue anchor 50 sleeves on the rotation driving body 6024, when the proximal end of the catheter 6022 is manipulated from the outside of the human body, movement or rotation of the rotation driving body 6024 may be achieved, and thereby effecting delivery or driving of the tissue anchori 50.

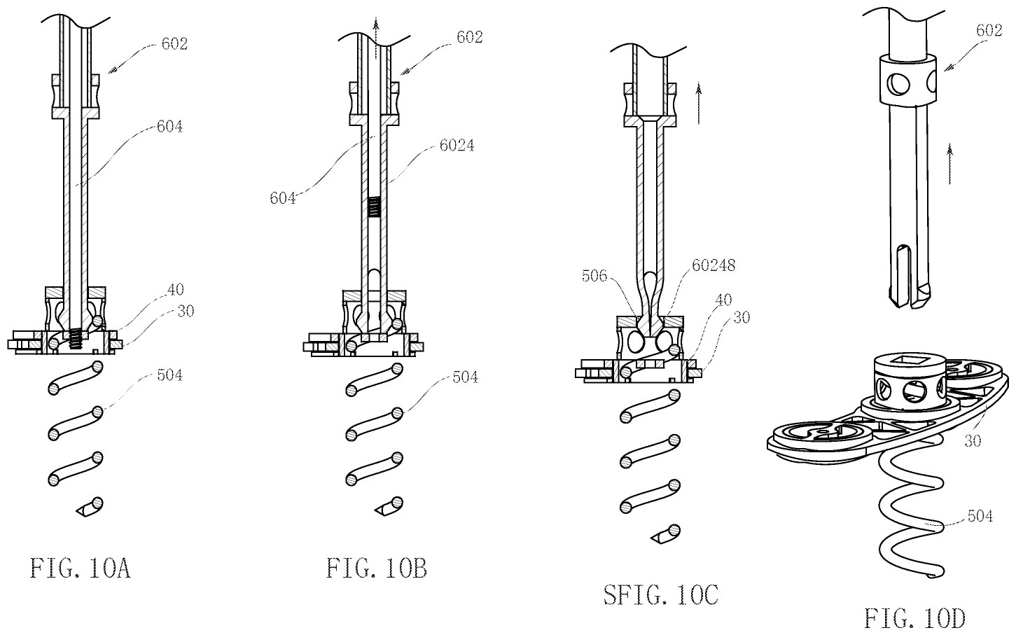

As shown in FIGS. 7A and 7B, the distal end external thread 6042 (see FIG. 6A) of the guide 604 may be detachably threadably engaged with the threaded hole 410 (see FIG. 4C) provided in the crossbar 408 of the rotatable portion 40 on the plate-shaped implant 30. The anchor deployment tool 602 may sleeve on the guide 604, and the anchor 50 may sleeve on the rotation driving body 6024 of the anchor deployment tool 602. The anchor 50 is axially movable between the catheter connection portion 606 and the tabs 60248. As shown in FIG. 7B, the guide 604 is inserted between the distal tabs 60248, thereby keeping the tabs 60248 separated at all times, thereby preventing the anchor 50 from disengaging from the rotation driving body 6024 towards the distal direction. As shown in FIG. 7A, the head portion 602 may apply a pushing force to the anchor 50 when the proximal end face of the head portion 502 of the anchor 50 is in contact with the catheter connection portion 606, the anchor 50 is moved towards the direction of the plate-shaped implant 30. In this way, in the event of bending of the guide 604, the anchor 50 can be smoothly brought to the position to be anchored by means of the pushing force of the anchor deployment tool 602 and the auxiliary guiding action of the rotation driving body 6024. As shown in FIGS. 10B to 10D, when the guide 604 is withdrawn from between the distal tabs 60248, the tabs 60248 may approach to the middle, thereby allowing the tabs 60248 to be separated from the anchor 50. In this way, the anchor deployment tool 602 can be effectively and reliably separated from the anchor 50.

The head portion 502 may be located anywhere between the catheter connection portion 606 and the tabs 60248 during the engagement of the anchor deployment tool 602 and the guide 604 to deliver the anchor 50. In some cases, the catheter connection portion 606 applies a pushing force to the head portion 502 to drive the anchor 50 to slide toward the plate-shaped implant 30. In other cases, the anchor 50 automatically slides toward the plate-shaped implant 30 under the guidance of the rotation driving body 6024 of the anchor deployment tool 602, and the tabs 60248 come into contact with the head portion 502 to limit the anchor 50 to continue to move towards the distal end. When the anchori deployment tool 602 is further moved in a direction toward the plate-shaped implant 30, the anchor 50 can continue to move closer to the plate-shaped implant 30. In some cases, the anchor deployment tool 602 may drive the anchor 50 to move to the proximal end via the tabs 60248.

The anchor 50 is delivered by the cooperation of the anchor deployment tool 602 and the guide 604. Since the rotation driving body 6024 is located between the anchor 50 and the guide 604 (in other words, the rotation driving body 6024 extends through the passage of the anchor 50), and the gap between the helical inner surface of the helical tissue coupling element 504 of the anchor 50 and the apex of the cross section of the rotation driving body may be set to be smaller, it is thus possible to ensure that the anchor 50 has good concentricity with the rotation driving body 6024. At the same time, the diameter of the guide 604 is matched with that of the longitudinal through hole 60242 of the rotation driving body 6024, which ensures that the guide 604 is concentric with the rotation driving body 6024, it is thus possible to ensure that the anchor 50 has good concentricity with the guide 604. In addition, the elongated rotation driving body 6024 increases the length available to guide the anchor 50, so that the rotation driving body 6024 functions not only to transmit torque to drive the anchor 50, but also to guide the anchor 50.

FIGS. 7A to 10D schematically illustrate an exemplary process of delivering an anchor 50, threading the anchor 50 into the tissue, and separating the anchor 50, by means of the cooperation of the anchor deployment tool 602 with the guide 604.

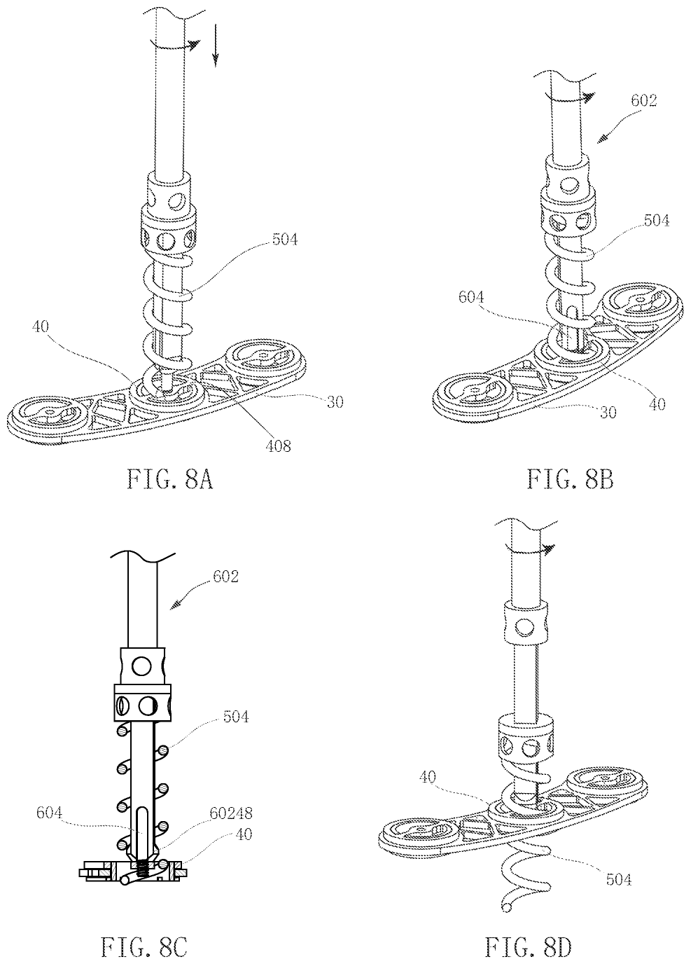

As shown in FIGS. 7A, 8A, in cooperation with the anchor deployment tool 602 and the guide 604, the anchor 50 is caused to slide towards a distal end direction along the elongated guide 604 by the pushing action of the catheter connection portion 606, until the distal end of the helical tissue coupling element 504 of the anchor 50 comes into contact with the proximal end surface (the upper surface in FIG. 8A) of the crossbar 408 of the rotatable portion 40. FIGS. 8A to 10D illustrate a process that the distal end of the helical tissue coupling element 504 contacts the upper surface of the crossbar 408 until the anchor deployment tool 602 and guide 604 are disengaged from the anchor 50.

As shown in FIG. 8A, the distal end of the helical tissue coupling element 504 of the anchor 50 has reached the upper surface of the rotatable portion 40. It is possible to continue to drive the anchor deployment tool 602 to advance along the guide 604 by manipulating the proximal end of the catheter 6022 while rotating the anchor deployment tool 602, thereby causing the helical tissue coupling element 504 of the anchor 50 to helically advance around the crossbar 408 of the rotatable portion 40 until the distal ends of the tabs 60248 reach the upper surface of the crossbar 408 (see FIGS. 8B, 8C). At this time, the distal end of the helical tissue coupling element 504 reaches at least the lower surface of the rotatable portion 40 (i.e., at least is in contact with the tissue). Alternatively, the distal end of the helical tissue coupling element 504 may also have entered the tissue at this time. As shown in FIGS. 8B, 8C, the distal ends of the tabs 60248 have reached the upper surface of the rotatable portion 40. At this time, as shown in FIG. 8D, the anchor deployment tool 602 is driven further to rotate, causing the helical tissue coupling element 504 to continue to helically advance around the crossbar 408. The anchor 50 will slide towards a distal end direction along the elongate rotation driving body 6024 of the anchor deployment tool 602, thereby further threading the anchor 50 into the tissue.

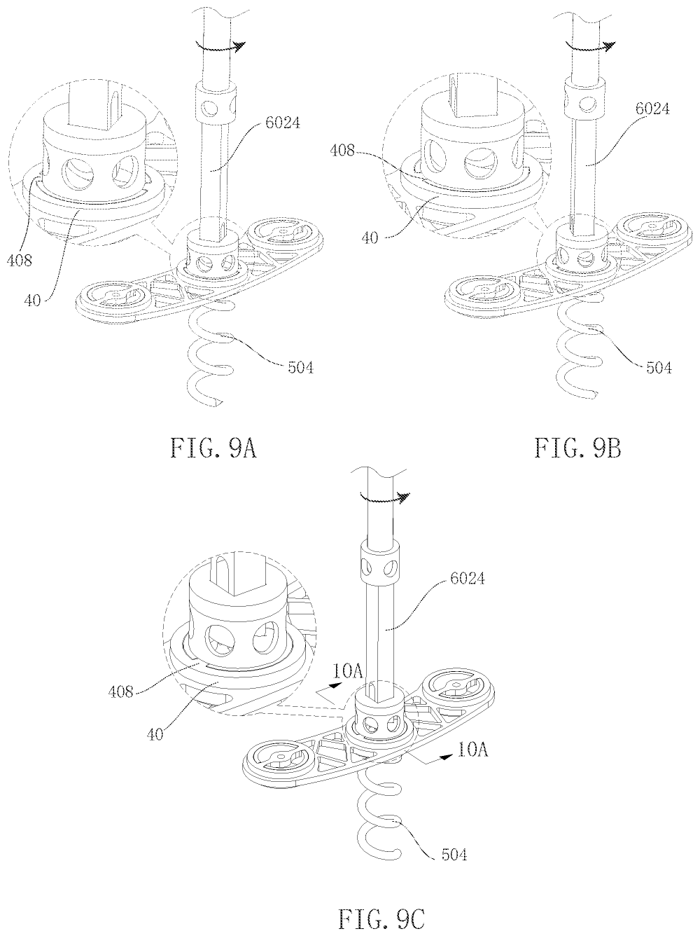

FIGS. 9A to 9C illustrate a schematic process of continuing to drive the anchor deployment tool 602 to rotate while the proximal end of the helical tissue coupling element 504 is in contact with the crossbar 408. As shown in FIG. 9A, the proximal end of the helical tissue coupling element 504 is brought into contact with the crossbar 408. As shown in FIG. 9B, the anchor deployment tool 602 continues to be driven to rotate, to cause the proximal end of the helical tissue coupling element 504 to come into contact with the crossbar 408 and rotate together with the anchor deployment tool 602. As shown in FIG. 9C, the anchor deployment tool 602 continues to be driven to rotate, to cause the helical tissue coupling element 504 to advance further into the tissue.

By comparing FIGS. 9A to 9C (see the circumferential position of the crossbar 408), it can be seen that after the proximal end of the helical tissue coupling element 504 is in contact with the crossbar 480, since the plate-shaped implant 30 is provided with a rotatable connection mechanism, the helical tissue coupling element 504 of the anchor 50 can be further rotated relative to the plate-shaped implant 30, thereby eliminating or reducing the gap created between the plate-shaped implant 30 and the tissue during the screwing-in of the anchor 50, the anchor 50 is fixed more securely, thereby effectively preventing the anchor 50 from being detached from the tissue.

FIGS. 10A to 10D illustrate the entire process of separation of the anchor deployment tool 602 from the anchor 50 and the plate-shaped implant 30. As shown in FIG. 10A, the anchor 50 has been anchored in place (i.e., the gap between the implant and the tissue has been eliminated or reduced). As shown in FIG. 10B, the guide 604 has been withdrawn between the distal tabs 60248 of the anchor deployment tool 602. At this time, the tabs 60248 are restored to the naturally separate state as shown in FIG. 10B. As shown in FIG. 10C, by manipulating the proximal end of the catheter to move the anchor deployment tool 602 towards a proximal direction, the tabs 60248 are brought inward toward each other by the opening wall of the non-circular engagement opening 506 of the head portion 502, so as to allow the tabs 60248 to pass through the opening 506. As shown in FIG. 10D, the tabs 60248 are separated from the anchor 50, i.e., the anchor deployment tool 602 is separated from the anchor 50.

Preferably, after the guide 604 is withdrawn between the distal tabs 60248 of the anchor deployment tool 602, the tabs 60248 are restored to an inward proximity to one another to facilitate passage of the tabs 60248 through the opening 506.

The distal end of the guide 604 remains substantially perpendicular to the plate-shaped implant when anchoring of the anchor 50 is performed in cooperation with the anchor deployment tool 602 and guide 604. Since the elongate rotation driving body 6024 extends within the channel of the anchor 50 during the anchoring process to guide the anchor 50 while driving the anchor 50, and the diameter of the guide 604 is adapted to the longitudinal through hole of the elongate rotation driving body 6024, thereby ensuring that the elongate driving body 6024, the guide 604 and the anchor 50 are substantially concentric, and this may ensure that the anchor deployment tool 602 screws the anchor 50 into the tissue substantially vertically.

Specific embodiments of the annuloplasty apparatus and the procedural apparatus according to the embodiments of the present disclosure have been described above with reference to the accompanying drawings. However, these descriptions are merely for purposes of describing the basic principles of the disclosure and applications thereof, and are not intended to limit the scope of the disclosure. The scope of the present disclosure is defined merely by the appended claims and their equivalents. Many different embodiments may be envisaged by those skilled in the art in view of the present disclosure.

1. An annuloplasty apparatus configured to be implanted into the body of a subject, the annuloplasty apparatus comprising:
a contractile bridging element;
a bar member connected to the contractible bridging element, the bar member being provided with a rotatable connection mechanism; and
a tissue anchor configured to secure the bar member to the annulus tissue by the rotatable connection mechanism, and comprising:
a head portion; and
a helical tissue coupling element, the proximal end of which is fixed to the head portion, wherein the helical tissue coupling element is configured to be driven into the annulus tissue by rotation,
wherein the rotatable connection mechanism is at least partially configured to be rotatable relative to the bar member, thereby allowing the tissue anchor to rotate further relative to the bar member when the proximal end of the helical tissue coupling element is in contact with the rotatable connection mechanism.

2. The annuloplasty apparatus according to claim 1, wherein, the rotatable connection mechanism comprises: a fixed portion that is fixedly provided with respect to the bar member; and a rotatable portion through which the tissue anchor is capable of fixing the bar member to the annulus tissue, wherein one of the rotatable portion and the fixed portion is provided with an annular circumferential groove, and the other is received in the annular circumferential groove in a rotatable manner with respect to the one.

3. The annuloplasty apparatus according to claim 2, wherein the bar member includes a first surface, a second surface opposite to the first surface and a circular through hole extending from the first surface to the second surface, and
wherein the rotatable portion comprises: a tubular body having a proximal end and a distal end, a top ring, and a bottom ring, the top ring and the bottom ring being disposed at the proximal end and the distal end of the tubular body, respectively, thereby defining the annular circumferential groove extending along outer circumference of the tubular body;
wherein the fixed portion is a portion of the bar member defining the circular through hole, and the portion is received in the annular circumferential groove.

4. The annuloplasty apparatus according to claim 3, wherein the rotatable portion further includes a connection portion disposed at one of the top ring, the tubular body, and the bottom ring, with the tissue anchor securing the bar member to the annulus tissue by the connection portion.

5. The annuloplasty apparatus according to claim 4, wherein the connection portion is a crossbar radially fixed to an inner peripheral wall of the top ring, or a proximal end face, or a distal end face of the top ring, and the proximal end face of the tubular body is provided with two notches that are opposed radially and are configured to receive the crossbar.

6. The annuloplasty apparatus according to claim 5, wherein the distal end face of the tubular body is provided with a plurality of notches configured to receive a plurality of projections, respectively, disposed radially inwardly on the inner peripheral surface of the bottom ring.

7. The annuloplasty apparatus according to claim 4, wherein the connection portion is a crossbar radially fixed to an inner peripheral wall of the bottom ring or a proximal end face or a distal end face of the bottom ring, and the distal end face of the tubular body is provided with two notches that are opposed radially and are used to receive the crossbar.

8. The annuloplasty apparatus according to claim 7, wherein the proximal end face of the tubular body is provided with a plurality of notches configured to receive a plurality of projections, respectively, disposed radially inwardly on the inner peripheral surface of the top ring.

9. The annuloplasty apparatus according to claim 5, wherein a threaded hole is provided in the center of the crossbar, which threaded hole is configured to be detachably engaged with the distal end external thread of a guide for guiding the tissue anchor.

10. The annuloplasty apparatus according to claim 3, wherein one of the top ring and the bottom ring is integrally formed with the tubular body.

11. The annuloplasty apparatus according to claim 2, wherein the bar member includes a first surface, a second surface opposite to the first surface and a circular through hole extending from the first surface to the second surface;
wherein the fixed portion includes a portion of the bar member defining the circular through hole, and
a top plate having a circular through hole, the top plate being secured to the first surface with its circular through hole substantially concentric with the circular through hole of the bar member, wherein the annular circumferential groove is provided in the inner peripheral surface of the circular through hole of the bar member and/or the top plate in the circumferential direction; and
the rotatable portion includes a circular ring rotatably received in the annular circumferential groove.

12. The annuloplasty apparatus according to claim 2, wherein the bar member includes a first surface, a second surface opposite to the first surface and a through hole extending from the first surface to the second surface, the fixed portion includes a split-type fixed base configured to be fixed within the through hole and defining a circular through hole, wherein the annular circumferential groove is provided in the inner peripheral surface of the circular through hole in the circumferential direction, and the rotatable portion includes a circular ring rotatably received in the annular circumferential groove of the split-type fixed base.

13. The annuloplasty apparatus according to claim 11, wherein the circular ring is provided with a connection portion, and the tissue anchor secures the bar member to the annulus tissue by the connection portion.

14. The annuloplasty apparatus according to claim 13, wherein the connection portion is a crossbar radially fixed to an inner peripheral wall of the circular ring or a proximal end face or a distal end face of the circular ring, wherein a threaded hole is provided in the center of the crossbar, which threaded hole is configured to be detachably engaged with the distal end external thread of a guide for guiding the tissue anchor.

15. The annuloplasty apparatus according to claim 12, wherein the distal end surface of the split-type fixed base is flush with the second surface of the bar member when secured within the through hole of the bar member.

16. A procedural apparatus for performing an annuloplasty, the procedural apparatus comprising:
a tissue anchor having a longitudinal center axis and being configured to define a passage extending through the tissue anchor along the longitudinal central axis, wherein the tissue anchor includes a helical tissue coupling element having a proximal end and a distal end, the helical tissue coupling element defining a portion of the passage of the tissue anchor;
an anchor deployment tool including:
a catheter having a catheter distal end; and
a rotation driving body having a proximal end, a distal end, and a longitudinal through hole extending from the proximal end to the distal end thereof, wherein the proximal end of the rotation driving body is connected to the catheter distal end; and
an elongate guide configured to be able to extend through the catheter and the longitudinal through hole of the rotation driving body,
wherein the rotation driving body is configured to guide the tissue anchor by extending within the passage of the helical tissue coupling element during anchoring of the tissue anchor to annulus tissue by rotating the tissue anchor.

17. The procedural apparatus according to claim 16, wherein the tissue anchor further includes a head portion fixed to the proximal end of the helical tissue coupling element and configured to define a non-circular engagement opening extending through the head portion along the longitudinal central axis, the non-circular engagement opening defining another portion of the channel of the tissue anchor,
wherein the rotation driving body has a non-circular cross section perpendicular to its longitudinal direction, wherein the non-circular cross section is configured to be adaptively and removably engaged with the non-circular engagement opening throughout the longitudinal length of the rotation driving body to drive the tissue anchor to rotate when the rotation driving body is rotated, thereby anchoring the tissue anchor to the annulus tissue while the tissue anchor is sliding in a distal end direction along the rotation driving body.

18. The procedural apparatus according to claim 17, wherein the distal end of the rotation driving body includes a furcation body, the radial outer side of the distal end of the furcation body is provided with tabs, wherein the furcation body is configured such that when the guide is inserted between the tabs, the tabs are pushed radially outward by the guide, thereby preventing the tabs from passing through the non-circular engagement opening of the head portion; and when the guide between the tabs is removed, the tabs return to the naturally separate state or a state of an inward proximity to one another, allowing the tabs to pass through the non-circular engagement opening of the head portion.

19. The procedural apparatus according to claim 18, wherein the anchor deployment tool further includes a catheter connection portion at the proximal end of the rotation driving body, the catheter connection portion is used for connecting the catheter distal end of the catheter, wherein the catheter connection portion is further configured to drive the tissue anchor to move distally on the rotation driving body along with the rotation driving body when the proximal end of the catheter is driven from the exterior of the subject to deliver the tissue anchor.

20. The procedural apparatus according to claim 19, wherein the length of the rotation driving body is configured such that when the distal ends of the tabs cannot be moved further towards the distal direction, the distal end of the helical tissue coupling element of the tissue anchor is in direct contact with the annulus tissue.

21. The procedural apparatus according to claim 18, wherein the furcation body is made of a shape memory material.

22. An annuloplasty system, comprising:
the annuloplasty apparatus according to claim 1, wherein the tissue anchor has a longitudinal center axis and is configured to define a passage extending through the tissue anchor along the longitudinal central axis, and the helical tissue coupling element defines a portion of the passage of the tissue anchor; and
an anchor deployment tool including:
a catheter having a catheter distal end; and
a rotation driving body having a proximal end, a distal end, and a longitudinal through hole extending from the proximal end to the distal end thereof, wherein the proximal end of the rotation driving body is connected to the catheter distal end; and
an elongate guide configured to be able to extend through the catheter and the longitudinal through hole of the rotation driving body, and to be detachably connected to the rotatable connection mechanism;
wherein the rotation driving body is configured to guide the tissue anchor by extending within the passage of the helical tissue coupling element during anchoring of the tissue anchor to annulus tissue by rotating the tissue anchor.

23. The annuloplasty system according to claim 22, wherein the tissue anchor further includes a head portion fixed to the proximal end of the helical tissue coupling element and configured to define a non-circular engagement opening extending through the head portion along the longitudinal central axis, the non-circular engagement opening defining another portion of the longitudinal channel of the tissue anchor,
wherein the rotation driving body has a non-circular cross section perpendicular to its longitudinal direction, wherein the non-circular cross section is configured to be adaptively and removably engaged with the non-circular engagement opening throughout the longitudinal length of the rotation driving body to drive the tissue anchor to rotate when the rotation driving body is rotated, thereby anchoring the tissue anchor to the annulus tissue while the tissue anchor is sliding in a distal end direction along the rotation driving body.

24. The annuloplasty system according to claim 23, wherein the distal end of the rotation driving body includes a furcation body, the radial outer side of the distal end of the furcation body is provided with tabs, wherein the furcation body is configured such that when the guide is inserted between the tabs, the tabs are pushed radially outward by the guide, thereby preventing the tabs from passing through the non-circular engagement opening of the head portion of the tissue anchor; and when the guide between the tabs is removed, the tabs return to the naturally separate state or a state of an inward proximity to one another, allowing the tabs to pass through the non-circular engagement opening of the head portion.

25. The annuloplasty system according to claim 24, wherein the anchor deployment tool further includes a catheter connection portion at the proximal end of the rotation driving body, the catheter connection portion is used for connecting the catheter distal end of the catheter, wherein the catheter connection portion is further configured to drive the tissue anchor to move distally on the rotation driving body along with the rotation driving body when the proximal end of the catheter is driven from the exterior of the subject to deliver the tissue anchor.

26. The annuloplasty system according to claim 25, wherein the length of the rotation driving body is configured such that when the distal ends of the tabs cannot be moved further towards the distal direction, the distal end of the helical tissue coupling element of the tissue anchor is in direct contact with the annulus tissue.
